SEQUENCE LISTING (9 sequences)

SEQ ID NO: 1 - protein, 129 aa, Homo sapiens
TALAGTRTAQGSGGGAGRGHGRRGRSRCSRKPLHVDFKELGWDDWIIAPLDYEAYHCEGLCDFPLRSHLEPTNHAIIQTLLNSMAPDAAPASCCVPARLSPISILYIDAANNVVYKQYEDMVVEACGCR

SEQ ID NO: 2 - DNA, 396 bp, Homo sapiens
  CDS (1)..(390)
atgactgctctggctggtactcgtactgctcaaggttctggtggtggcgctggccgtggtcacggtcgtcgtggtcgttctcgttgttctcgtaaaccgctgcacgttgacttcaaagaactgggttgggacgactggatcatcgctccgctggactacgaagcataccactgtgaaggtctgtgcgacttcccgctgcgttctcacctggaaccgaccaaccacgctatcatccagaccctgctgaactctatggcaccggacgctgcaccggcttcttgttgtgttccggcacgtctgtctccgatctccatcctgtacatcgacgctgcaaacaacgttgtttacaaacagtacgaagacatggttgttgaagcatgcggttgccgttaataa

SEQ ID NO: 3 - protein, 120 aa, Homo sapiens
TAFASRHGKRHGKKSRLRCSKKPLHVNFKELGWDDWIIAPLEYEAYHCEGVCDFPLRSHLEPTNHAIIQTLMNSMDPGSTPPSCCVPTKLTPISILYIDAGNNVVYKQYEDMVVESCGCR

SEQ ID NO: 4 - protein, 120 aa, Homo sapiens
APLATRQGKRPSKNLKARCSRKALHVNFKDMGWDDWIIAPLEYEAFHCEGLCEFPLRSHLEPTNHAVIQTLMNSMDPESTPPTCCVPTRLSPISILFIDSANNVVYKQYEDMVVESCGCR

SEQ ID NO: 5 - protein, 32 aa, Homo sapiens
SHLEPTNHAIIQTLLNSMAPDAAPASCCVPAR

SEQ ID NO: 6 - protein, 32 aa, Artificial Sequence
  Description of Artificial Sequence Synthetic polypeptide
SHLEPTNHAIIQTLLNSXAPDAAPASCCVPAR

SEQ ID NO: 7 - protein, 13 aa, Homo sapiens
QYEDMVVEACGCR

SEQ ID NO: 8 - protein, 13 aa, Artificial Sequence
  Description of Artificial Sequence Synthetic peptide
QYEDXVVEACGCR

SEQ ID NO: 9 - protein, 130 aa, Homo sapiens
MTALAGTRTAQGSGGGAGRGHGRRGRSRCSRKPLHVDFKELGWDDWIIAPLDYEAYHCEGLCDFPLRSHLEPTNHAIIQTLLNSMAPDAAPASCCVPARLSPISILYIDAANNVVYKQYEDMVVEACGCR

(19) United States
(12) Patent Application Publication
(10) Pub. No.: US 2010/0004175 A1
(43) Pub. Date: Jan. 7, 2010

(54) NOVEL BMP-12-RELATED PROTEINS AND METHODS OF THEIR MANUFACTURE

(76) Inventors: Tanya Shang, Swampscott, MA (US); Ramesh Matur, Hyderabad (IN); Heather Degruttola, Newton, NH (US)

Correspondence Address: WYETH/FINNEGAN HENDERSON, LLP, 901 NEW YORK AVENUE, NW, WASHINGTON, DC, 20001-4413, US

(21) Appl. No.: 12/480,327

(22) Filed: Jun. 8, 2009

Related U.S. Application Data
(60) Provisional application No. 61/059,870, filed on Jun. 9, 2008.

(51) Int. Cl. A61K 38/16; C07K 14/435; C07K 5/00; C07K 7/06; C07K 7/08; C12P 21/06; C07H 21/04

(52) U.S. Cl. 514/12; 530/350; 530/331; 530/330; 530/329; 530/328; 530/327; 530/326; 530/325; 530/324; 435/69.1; 536/23.1

(57) ABSTRACT

The invention provides novel BMP-12-related proteins, including methods of their manufacture. The proteins include substituted, truncated and substituted-truncated BMP-12-related proteins. The substituted BMP-12-related proteins contain a substitution at one or more oxidation-sensitive methionine residues with non-methionine residues, such as norleucine. The substituted BMP-12-related proteins exhibit normal bioactivity and enhanced resistance to oxidation, relative to the unsubstituted protein. The truncated BMP-12-related proteins exhibit enhanced activity.

This application claims the benefit of the earlier filing date of U.S. Provisional Patent Application No. 61/059,870, filed Jun. 9, 2008, which is incorporated by reference in its entirety.

The invention relates to the field of peptide growth factors. In particular, the invention relates to novel BMP-12-related proteins, which have tendon and or ligament-like tissue inducing activity, and methods of their manufacture.

Members of the transforming growth factor-beta (TGF-62 ) superfamily possess physiologically important growth-regulatory and morphogenetic properties (Kingsley et al., Genes Dev. 8:133-146 (1994); Hoodless et al., Curr. Topics Microbiol. Immunol. 228:235-272 (1998)). Bone morphogenetic proteins (BMPs) are members of the TGF-β superfamily of growth and differentiation factors (Rosen et al., Principles of Bone Biology 2:919-928 (2002)). Some of the first evidence that BMPs existed was demineralized bone's ability to induce new bone when implanted into muscle (Urist et al., Science 150:893-99 (1965)). BMPs were subsequently biochemically purified from demineralized bone (Wang et al., PNAS 85: 9484-9488 (1988)) and cloned by hybridization of radiolabeled oligonucleotides designed from peptide fragments of the purified proteins (Wozney et al., Science 242:1528-1534 (1988)). Cloned BMPs have been recombinantly expressed and retain their function. For example, recombinant mature BMP-2 (amino acids 283-396) expressed in E. coli exhibits bone stimulating activity both in vitro (Ruppert et al., Euro. J. Biochem. 237:295-302 (1996)) and in vivo (Kübler et al., Int J. Oral Maxillofacial Surgery 27:305-09 (1998)).

Additional BMPs were cloned by screening for homologues of known BMPs, and have been shown to possess a wide range of activities, including induction of the growth and differentiation of bone, connective, kidney, heart, and neuronal tissues (Rengachary, Neurosrug. Focus 13(6):1-6 (2002)).

BMP-12-related proteins, which include BMP-12, BMP-13, and MP-52 (also known as GDFs 7, 6, and 5, respectively) are a sub-genus of BMPs which possess tendon and/or ligament-forming activity (Storm et al., Nature 368:639-643 (1994); Wolfman et al., J. Clin. Invest. 100:321-330 (1997); and International Publication No. WO 95/16035). In vivo, the proteins are synthesized as large pre-proproteins and are proteolytically processed to produce mature, bioactive, dimeric proteins containing two subunits, each approximately 120-130 residues long. The mature form of BMP-12 can be produced recombinantly in bacterial cells such as E. coli.

Common sites of tendon and/or ligament injury include the anterior cruciate ligament (Laurencein et al., Annu. Rev. Biomed. Eng. 1:19-46 (1999)), Achilles' tendon (Mazzone and McCue, Am. Fam. Physician 65:1805-10 (2002)), rotator cuff, and flexor tendon in the hand (Boyer et al., J. Hand Ther. 18:80-85 (2005)). Other sources of maladies in tendon or ligament-like tissue include injury, failure, or congenital defects in the ligament-like fascia tissue, which penetrates, supports and surrounds most organs and tissues of the body. Damage to the fascia tissue can result in hernias or organ prolapse, for example bladder, uterine, or rectal prolapse.

In addition to the ability of BMP-12-related proteins to affect ectopic growth of tendon and/or ligament-like tissue (see, WO 95/16035; Wolfman et al., 1997; and Helm et al., J. Neurosurg. 95:298-307 (2001)), BMP-12 and its related proteins have been shown to augment repair of these tissues. For example, BMP-12 improved repair in animal models of rotator cuff (Archambault et al., 5^{th} Comb. Mtg. Ortho. Res. Soc. Canada, USA, Japan, and Europe Podium No: 128, (2004)), patellar tendon (Archambault et al., 5^{th} Comb. Mtg. Ortho. Res. Soc. Canada, USA, Japan, and Europe Poster No: 197, (2004)), and flexor profundus tendon (Lou et al., J. Ortho. Res. 19:1199-1202 (2001)). Similarly, MP-52 (GDF-5) stimulated healing in an Achilles' tendon defect (Rickert et al., Growth Factors 19:115-26 (2001)).

Native hBMP-12 contains methionine residues at positions 84 and 121 of the mature protein. These two methioines are conserved in most species of BMP-12 and also among the human BMP-12-related proteins—BMP-12, BMP-13, and MP-52—suggesting that these residues play an important functional role in the protein. However, without careful process control, these methionines are particularly susceptible to oxidation during large-scale production of BMP-12-related proteins, resulting in deactivation of the protein. Accordingly, there is a need for BMP-12-related proteins that are amenable to large-scale production and maintain their tendon and/or ligament-like tissue inducing activity.

The present invention provides novel BMP-12 and related proteins with increased resistance to oxidation inactivation. The BMP-12-related proteins of the invention are particularly amenable to high throughput production in order to meet the expanding need for these protein-based therapeutics. The invention is based, in part, on the surprising discovery that a mature BMP-12 protein having a non-methionine residue substituted for one or more native methionine residues (“substituted BMP-12-related protein”) not only exhibited increased resistance to inactivation by oxidation, but also maintained its in vitro activity. This is particularly surprising in view of the fact that these residues are highly conserved and thus, generally thought to be important to the activity of the protein.

Thus, in one aspect, the invention provides a substituted BMP-12-related protein able to induce the formation of tendon and/or ligament-like tissue. The substituted BMP-12-related protein has at least one amino acid substitution at a residue corresponding to the methionines of a mature BMP-12-related protein. In some embodiments, a substitution may be at a residue corresponding to methionine 84 of SEQ ID NO:1. In other embodiments, a substitution may be at a residue corresponding to methionine 121 of SEQ ID NO:1. In still further embodiments, there may be substitutions at residues corresponding to both methionine 84 and 121 of SEQ ID NO:1.

In some embodiments, a methionine residue of a substituted BMP-12-related protein is substituted with an amino acid chosen from norleucine, leucine, isoleucine, valine, alanine, or phenylalanine. In more particular embodiments a methionine residue is substituted with norleucine, leucine, or isoleucine. In still more particular embodiments, a methionine residue is substituted with norleucine. Substituted BMP-12-related proteins with substitutions of two or more methionines may have the same residues substituted at each of the methionines, or different residues substituted at each of the methionines.

In certain embodiments, the substituted BMP-12-related protein comprises a sequence that is at least 90% identical to any one of SEQ ID NOs:1, 3, or 4 and can induce tendon and/or ligament-like tissue. In some embodiments, the BMP-12-related protein is BMP-12. In other embodiments, the BMP-12-related protein is BMP-13. In still other embodiments, the BMP-12-related protein is MP-52. In some embodiments, the substituted BMP-12-related proteins of the invention include at least one truncated subunit, i.e., one monomer of the dimeric protein, with an N-terminal truncation of 1 to 27 amino acids in length (“substituted-truncated BMP-12-related protein”). In particular embodiments, the N-terminal truncation is at most 22, e.g., 18 or 7 amino acids in length. In some embodiments, the invention provides a BMP-12-related protein having at least one truncated subunit but does not contain any substitutions at the residues corresponding to methionine 84 or 121 of SEQ ID NO:1 (“truncated BMP-12-related protein”).

In some embodiments, the substituted BMP-12-related proteins of the invention are part of a composition. In certain embodiments, the composition further comprises a BMP-12-related protein having methionine at residues corresponding to methionine 84 and 121 of SEQ ID NO:1 that can induce tendon and/or ligament-like tissue formation. In some embodiments, the composition further comprises a suitable pharmaceutical carrier.

In certain embodiments, the substituted BMP-12-related protein may make up at least 0.1%, 1%, 5%, 10%, 15%, 20%, 30%, 40%, 50%, 75%, 80%, 85%, 90%, 95%, 99%, 99.9%, or more, of the BMP-12-related proteins in the composition. In certain embodiments, the composition is produced by fermentation in bacterium. In some embodiments, the bacterium is cultured in conditions selected from the group consisting of limited methionine, limited leucine, excess norleucine, and combinations thereof.

In another aspect the invention provides methods of treating a tendon or ligament defect in a subject comprising administering an effective amount of the pharmaceutical compositions of the invention.

In another aspect, the invention provides nucleic acids encoding the substituted BMP-12-related proteins of the invention. In one embodiment, the nucleic acid comprises a sequence that is at least 90% identical to nucleotides 4-390 of SEQ ID NO:2.

In certain embodiments, the nucleic acid encodes a substituted BMP-12-related protein where a methionine residue is substituted with an amino acid selected from the group consisting of leucine, isoleucine, valine, alanine, or phenylalanine. In more particular embodiments a methionine residue is substituted with leucine or isoleucine. In certain embodiments, the nucleic acids provided by the invention are contained in a vector or host cell. In particular embodiments, the host cell is a bacterium. In more particular embodiments, the bacterium is E. coli.

The accompanying drawings, which are incorporated in and constitute a part of this specification, illustrate several embodiments of the invention and together with the description, serve to explain the principles of the invention.

### BRIEF DESCRIPTION OF THE SEQUENCES

SEQ ID NO:1 is an amino acid sequence of mature human BMP-12.

SEQ ID NO:2 is a nucleic acid sequence encoding a mature human BMP-12. This sequence includes an “atg” start codon and two “taa” stop codons that do not encode residues present in the mature protein. A translation of this sequence is provided in SEQ ID NO:9.

SEQ ID NO:3 is an amino acid sequence of mature human BMP-13.

SEQ ID NO:4 is an amino acid sequence of mature human MP-52.

SEQ ID NOs:5 and 6 are sequences of BMP-12 T10 peptides.

SEQ ID NOs:7 and 8: are sequences of BMP-12 T12 peptides.

### BRIEF DESCRIPTION OF THE DRAWINGS

FIGS. 1A-1B shows the reducing RP-HPLC profiles of BMP-12 monomers with (FIG. 1B) or without (FIG. 1A) substituted species. FIG. 1A discloses ‘TALA’ as residues 1-4 of SEQ ID NO: 1 and ‘CGCR’ as residues 126-129 of SEQ ID NO: 1.

FIGS. 2A-2D show reducing RP-HPLC profiles of purified BMP-12 monomers (FIGS. 2A, 2B) and unpurified BMP-12 monomers present in the solubilized inclusion body (sIB) (FIGS. 2C, 2D) of batches with (FIGS. 2B, 2D) or without (FIGS. 2A, 2C) substituted species. “DS” refers to drug substance (purified BMP-12).

FIGS. 3A-3B are peptide maps of BMP-12 monomers from lot 174 (FIG. 1B, containing substituted species) and lot 148 (FIG. 1A, without substituted species). The new peaks in lot 174 are shown by dotted lines. Note: cbm: carbamylation; ox: oxidation; d: deamidation.

FIG. 4 shows the sequence of a mature human BMP-12 monomer (SEQ ID NO: 1), including the trypsin digestion products. Alternating string of all capital or all lower-case residues correspond to distinct tryptic peptides.

FIGS. 5A-5B show MS/MS fragmentation spectra of the T10 peptide for BMP-12 batches not containing (FIG. 5A) or containing (FIG. 5B) substituted species. FIGS. 5A and 5B disclose SEQ ID NOS 5-6, respectively, in order of appearance.

FIGS. 6A-6B show MS/MS fragmentation spectra of the T12 peptide for BMP-12 batches not containing (FIG. 6A) or containing (FIG. 6B) substituted species. FIGS. 6A and 6B disclose SEQ ID NOS 7-8, respectively, in order of appearance.

FIG. 7 is a fitted semi-logarithmic plot that shows the relative fluorescent units (RFUs) from a cell-based BMP-responsive element luciferase (BRE-luc) reporter as a function of rhBMP-12 concentration for batches with (174) and without (002) significant levels of substituted species.

FIGS. 8A-8H show reducing RP-HPLC profiles of monomers of wild-type BMP-12 (<5% per-site substitution) treated with varying levels of peracetic acid (PAA). 2ox: both methionine residues oxidized; 1ox: 1 of 2 methionine residues oxidized; 0ox: unoxidized.

FIG. 9 is a picture that shows a silver-stained SDS-PAGE, tricine gel of BMP-12 (dimers, <5% per-site substitution) treated with varying levels of PAA.

FIG. 10 is a plot that shows specific activity (as determined by BRE-luc bioassay) versus total percent oxidized species (the sum of singly- and doubly-oxidized monomer species) as measured by reducing RP-HPLC (FIG. 8) for highly purified BMP-12 (dimer, <5% per-site substitution) treated with varying levels of PAA. A least-squares regression line is included on the plot.

FIG. 11 is a plot that shows the percentage peak area on a RP-HPLC profile corresponding to doubly oxidized BMP-12 as a function of PAA concentration for samples with high (25-40% per site) and low (<5%) levels of substitution.

FIG. 12 is a plot that shows the percent control (untreated) activity of BMP-12 as measured in a BRE-luc bioassay for a batch of BMP-12 with a low (<5%) rate of substitution and a pool of batches of BMP-12 with high (25-40%) rates of substitution, as a function of PAA/BMP-12 molar ratio.

FIGS. 13A-13D are plots that show the results of non-reducing SDS-CE of buffer alone (FIG. 13A), batches containing (FIGS. 13C, 13D), and not containing (FIG. 13B) a truncated dimeric BMP-12 species. A 10 kDa internal standard is noted. The arrows show the new pre-peak (FIGS. 13C, 13D). IRM#1 is a reference material.

FIG. 14 shows a nanoESI QTOF MS/MS spectrum of a BMP-12 truncated monomer corresponding to ²³RGR . . . GCR¹²⁹ of the mature BMP-12. FIG. 14 discloses SEQ ID NO: 1.

FIG. 15 is a picture that shows an SDS-PAGE of BMP-12 treated with tryspin at various enzyme to substrate ratios.

FIG. 16 shows a fitted semi-logarithmic plot that shows the relative fluorescent units (RFUs) from a cell-based BMP-responsive element luciferase (BRE-luc) reporter as a function of rhBMP-12 concentration for samples with varying degrees of trypsin-induced truncation. The inset is a picture of an SDS-PAGE of the samples used in the assay, showing the degree of truncation present in each sample and estimated potency of each sample, relative to the un-truncated control.

FIG. 17 shows a multiple sequence alignment of the mature sequences of human BMP-12, BMP-13, and MP-52.

### EXEMPLARY EMBODIMENTS

A “BMP-12-related protein” is a dimeric protein that has tendon and/or ligament-like tissue inducing activity and contains two disulfide-linked monomeric subunits, which comprise a sequence that is at least 70%, 80%, 90%, 95%, 97%, 98%, 99%, or more identical at the amino acid level to the sequence of a mature BMP-12, BMP-13, or MP-52 (also known as GDFs 7, 6, and 5) protein. The present invention provides substituted, truncated, and substituted and truncated (“substituted-truncated”) BMP-12-related proteins and methods of their manufacture. These novel BMP-12-related proteins exhibit normal bioactivity and physical characteristics, but exhibit increased resistance to inactivation by oxidation, particularly during large-scale production.

In some embodiments, BMP-12-related proteins can include additional modifications including, e.g., carbamylation. Accordingly, a “carbamylated BMP-12-related protein” contains at least one carbamylated subunit. In some embodiments, a carbamylated BMP-12-related protein contains 2 carbamylated subunits. Carbamylation of BMP-12-related proteins occurs during purification when the proteins are incubated with high levels of urea. The urea helps to solubilize inclusion bodies, which contain the BMP-12-related proteins extracted from E. coli. Carbamylation does not appear to affect BMP-12-related protein activity. Any of the substituted, truncated, or substituted-truncated BMP-12-related proteins of the invention discussed herein may also be carbamylated.

### BMP-12-Related Proteins and Truncated BMP-12-Related Proteins

A “truncated BMP-12 related protein” has an N-terminal truncation of at least 1, 3, 5, 7, 10, 15, 18, 20, 21, 22, 23, 24, 25, 26, 27, or more residues from the N terminus of at least one subunit of the dimeric protein. In some embodiments, a truncated BMP-12-related protein contains one truncated subunit. In other embodiments, both subunits of the BMP-12-related protein are truncated. In these embodiments, the truncated subunits may be, but need not be, identical in length or sequence. In some embodiments, the truncation begins at a residue corresponding to the N-terminus of the mature form of a BMP-12-related protein subunit. In particular embodiments, the truncation begins at a residue corresponding to amino acid number 1 of SEQ ID NO:1, 3, or 4.

Thus, in certain embodiments, a truncated BMP-12-related protein contains a subunit comprising residues corresponding to amino acids 28-128, 28-129, 23-129, 22-129, 19-129, 8-129, 7-129, or 1-129, of SEQ ID NO:1; or 28-119, 28-120, 23-120, 19-120, 14-120, 13-120, 8-120, 7-120, 6-120, or 1-120 of SEQ ID NO:3 or 4. By “residue corresponding to” it is meant the residue which most closely plays the same functional and or structural role as the reference residue. This is determined by means known in the art, including sequence alignments, such as visual inspection, Smith-Waterman, BLAST, Markov models, or ClustalW. When comparing two sequences by homology, it is to be understood that the percent homology is over the length of the shorter sequence. For example, if a BMP-12-related protein has a ten residue N-terminal truncation and is 90% identical to SEQ ID NO:1, then 90% of the residues in the truncated protein correspond to SEQ ID NO:1. In certain embodiments, the BMP-12 related protein is at least 50, 60, 70, 80, 90, 100, 105, 110, or 115 residues in length. Any of the truncated BMP-12-related proteins provided by the invention may contain any of the methionine substitutions described below for substituted BMP-12-related proteins.

BMP-12-related proteins have been identified in numerous species, including, for example, human, macaque, mouse, and rat. As is known in the art, these sequences can be used to guide the preparation of additional substituted BMP-12-related proteins. Residues or motifs that are preserved among BMP-12-related proteins will tend to be important for their tendon and/or ligament-like tissue forming activity, while residues and motifs that differ between these proteins can likely be modified without destroying the tendon and/or ligament-like tissue forming activity of the protein. See, for example, Table 1, which lists the National Center for Biotechnology Information (NCBI) Entrez GeneID, and reference protein accession numbers (RefSeq) for BMP-12-related proteins from several species. These GeneIDs may be used to retrieve publicly-available annotated mRNA or protein sequences from the NCBI website, for example, at the following uniform resource locator (URL): http://www.ncbi.nlm.nih.gov/sites/entrez?db=gene. The information associated with these GeneIDs, including reference sequences and their associated annotations, are all incorporated by reference.

TABLE 1

| | BMP-12 | | BMP-13 | | MP-52 | |
|---|---|---|---|---|---|---|
| Species | Gene ID | Protein | Gene ID | Protein | Gene ID | Protein |
| Homo sapiens | 151449 | NP_878248 | 392255 | NP_001001557 | 8200 | NP_000548 |
| Bos taurus | 286859 | XP_616701 | | | 539559 | XP_588072 |
| Canis lupus familiaris | 482989 | XP_540103 | | | 485850 | XP_542974 |
| Danio rerio | 30642 | XP_694563 | | | | |
| Equus caballus | | | | | 100034228 | NP_001075989 |
| Gallus gallus | | | 428655 | | 374249 | NP_989669 |
| Macaca mulatta | 702462 | XP_001096970 | 701126 | XP_001090825 | 705432 | XP_001099702 |
| Mus musculus | 238057 | NP_038555 | 242316 | NP_038554 | 14563 | NP_032135 |
| Pan troglodytes | 470322 | | | | 458202 | XP_001164592 |
| Rattus norvegicus | 252833 | XP_345647 | 252834 | NP_001013056 | 252835 | XP_001066344 |
| Xenopus laevis | | | 399144 | NP_001083833 | | |
| Xenopus tropicalis | | | 548831 | NP_001016077 | | |

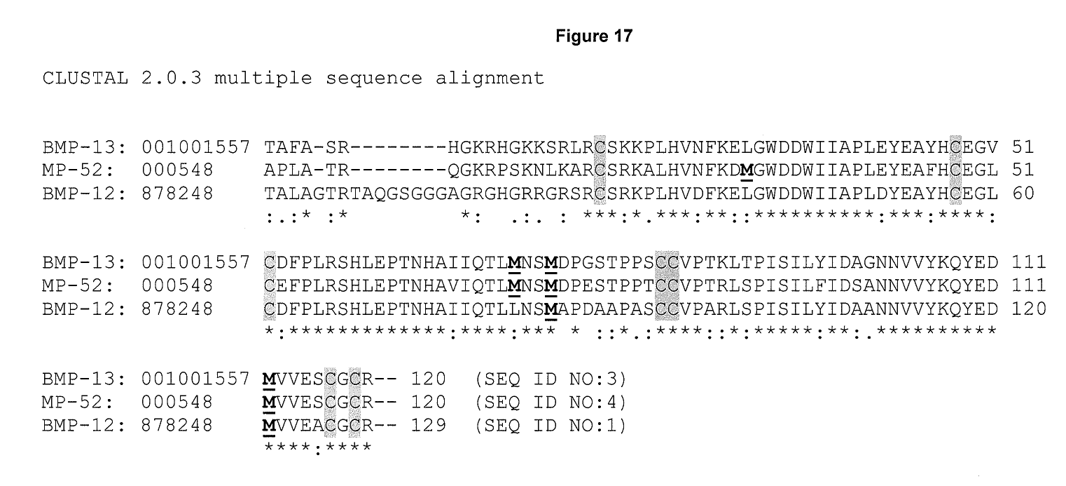

In addition, FIG. 17 provides a multiple sequence alignment of mature human BMP-12, BMP-13, and MP-52 proteins. The conserved cysteine residues corresponding to the cystine knot motif are highlighted while methionines are underlined and in bold. The indicated sequences are the NCBI RefSeq identifiers for the full-length pre-propeptides.

### Substituted BMP-12-Related Proteins

A “substituted BMP-12-related protein” has at least one residue corresponding to methionine residue 84 or 121 of SEQ ID NO:1 replaced with a non-methionine residue and retains tendon and/or ligament-like tissue forming activity. These substitutions may exist in one or both subunits of a BMP-12-related protein dimer. Accordingly, in certain embodiments, a substituted BMP-12-related protein has at least 1, 2, 3, or 4 non-methionine substitutions at these sites. When a BMP-12-related protein subunit contains additional methionines, these may optionally be substituted with a non-methionine residue. This encompasses from 1 to 2n substitutions, where “n” is the total number of methionines in a mature protein subunit (monomer). For example, a mature BMP-13 monomer has 3 native methionines: M75 and M112 of SEQ ID NO:3, which correspond to M84 and M121 of SEQ ID NO:1, respectively, and M72, which corresponds to L81 in SEQ ID NO:1. A mature MP-52 monomer has four native methionines: M75 and M112 of SEQ ID NO:4, which correspond to M84 and M121 of SEQ ID NO:1, respectively and M31 and M72 of SEQ ID NO:4, which correspond to L40 and L81 of SEQ ID NO:1. In the substituted BMP-12-related proteins of the invention, any or all of these methionines may be substituted with a non-methionine amino acid residue.

The amino acid residues substituted for methionine can include any of the 19 typical, naturally-occurring, non-methionine amino acids; any non-typical amino acids (for example, norleucine or norvaline); and amino acid analogs, derivatives, and modifications, so long as the substitution retains the protein's tendon and/or ligament-like tissue forming activity. In certain embodiments, the substitutions are selected from the group consisting of norleucine, leucine, isoleucine, valine, alanine, and phenylalanine. In more particular embodiments the substitutions are selected from the group consisting of norleucine, leucine, and isoleucine. In some embodiments one or more methionines in the BMP-12-related protein is substituted with norleucine.

### Biological Activity

Various methods for measuring the activity of BMP-12-related proteins are known in the art. These include cell-based assays, where a BMP-12-related protein changes an observable phenotype of cells, for example, affecting the morphological changes associated with tendon and/or ligament-like tissue in a suitable host cell or the inhibition of myoblast differentiation in mouse L6 cells (Inada et al., Biochem Biophys. Res. Comm. 222:317-22 (1996); shown for BMP-12). Another modality for detecting tendon and/or ligament-like tissue inducing activity is ectopic implantation. There, a capsule containing a BMP-12-related protein is implanted into a host animal for 1 to 2 weeks, recovered, and the capsule contents are evaluated histologically for the presence of, for example, tendon and/or ligament-like tissue (U.S. Pat. No. 6,150,328, Example III; Sampath and Reddi, Proc. Natl. Acad. Sci. U.S.A. 80:6591-6595 (1983)).

BMP-12-related protein activity can also be detected by monitoring the expression (that is, transcription or translation) of reporter molecules. This includes a cell-based BMP-response element-luciferase (BRE-luc) reporter construct (for a discussion of BREs, see Kusanagi et al., Mol. Bio. Cell 11:555-65 (2000)) or a characteristic BMP-12-related protein-induced expression profile in a BMP-12 responsive cell. U.S. patent application Ser. No. 12/393,628, filed Feb. 26, 2009, incorporated by reference, teaches additional methods of detecting BMP-12 (and related) protein activity in a cell-based assay. The methods include detecting and/or measuring the level of BMP-12-related-activity-markers, including thrombospondin-4 (THBS4, Homo sapiens GeneID 7060), by calculating a dose-response curve to a test sample containing, for example, BMP-12.

### Methods of Producing Novel BMP-12-Related Proteins

The substituted, truncated, or substituted-truncated BMP-12-related proteins of the invention can be produced by a variety of means known in the art, including, e.g., by controlling fermentation conditions before and/or during protein synthesis to produce spontaneous substitutions, genetic engineering, chemical synthesis, and enzymatic treatment.

Fermentation conditions that affect substitution at methionine residues include limited methionine, limited leucine, excess norleucine (for example, relative to methionine), and combinations thereof. Norleucine is a methionine analog, where a carbon atom replaces the single sulfur atom of methionine. It is theorized that norleucine is a low-affinity (relative to methionine) substrate for methionyl tRNA synthetase and the relative abundance of these two amino acids can affect rates of substitution by mass action. For example, excess norleucine relative to methionine can increase the rate of norleucine substitution.

Accordingly, in some embodiments, substituted BMP-12-related proteins can be produced in fermentation in conditions where norleucine is in molar excess of methionine. Norleucine, or another suitable, oxidation-resistant methionine analog, may be in at least 1.1, 1.2, 1.5, 1.8, 2,4, 8, 10, 20, 40, 50, 80, 100, 200, 400, 500, 800, 1000-fold, or more, molar excess relative to methionine. Norleucine may be added to the fermentation medium before or during protein synthesis. Alternatively or additionally, one or more norleucine precursors can be added to the fermentation medium before or during protein synthesis.

Leucine abundance can affect the rate of norleucine synthesis because the leucine-synthetic pathway is responsible for norleucine production (Kisumi et al., Appl. Envir. Microbiol., 34:135-138 (1977) and Kisumi et al., J. Biochem. 80:333-330 (1976)). For example, when leucine is limited, the leucine biosynthetic pathway is activated and norleucine will be synthesized. Conversely, when the leucine biosynthetic pathway is inactive (e.g., due to excess leucine in the growth medium), norleucine synthesis is reduced or discontinued.

Thus, in some embodiments, the cell may be grown under conditions known to favor activation of the leucine biosynthetic pathway, e.g., growth in medium with no, low, or limited leucine. For example, there may be at least 50%, 80%, 90%, 99%, or at least 1, 2, 5, 10, 20, 40, 100, 500-fold less leucine than under standard growth conditions. Leucine concentrations in some standard bacterial growth conditions may be about 30-120 mg/L, e.g., about 60 mg/L. In some embodiments, the fermentation medium contains no supplemental leucine. In some embodiments, the cells are grown for a period of time to diminish or deplete the available pool of free leucine before protein synthesis. For example, a growth medium containing an amino acid source, e.g., yeast extract or protein hydrolysate, can be depleted of amino acids by, for example, extending the growth phase of the host cells before inducing protein synthesis. In some embodiments, the host cell may have elevated expression levels of one or more leucine biosynthetic genes, relative to a wild-type host cell, e.g., resulting in constitutive activation of the leucine biosynthetic pathway, e.g., due to derepression.

In some embodiments, the host cell may be grown under conditions of no, low, or limited methionine. For example, there may be at least 50%, 80%, 90%, 99%, or at least 1, 2, 5, 10, 20, 40, 100, 500-fold less methionine than under standard growth conditions. Methionine concentrations in some standard bacterial growth conditions may be about 10-40 mg/L, e.g., about 20 mg/L. In some embodiments, the fermentation medium contains no supplemental methionine. In some embodiments, the cells are grown for a period of time to diminish or deplete the available pool of free methionine before protein synthesis. In some embodiments, the host cell may produce low levels of or no methionine, e.g., the cell is a methionine auxotroph. In more particular embodiments, the host cell may have reduced, low, or no expression of one or more methionine biosynthetic genes, e.g., methionine synthase, relative to wild-type host cells.

Certain fermentation conditions are known to affect spontaneous replacement of methionine with norleucine in a protein and can be used to produce the substituted BMP-12-related proteins of the invention. These include, for example, fermentation in culture medium with a 100 fold excess of norleucine to methionine: 200 mg/L of norleucine and 2 mg/L methionine, (Anfisen and Corley J. Biol. Chem. 244:5149-52 (1969), showing production of 15% fully-substituted recombinant staphylococcal nuclease in a Staphylococcus aureus methionine auxotroph). Another culture medium with an altered methionine/norleucine ratio is (g/liter): 6KH₂PO₄, 18.3K₂HPO₄, 4(NH₄)₂SO₄, 0.4MgS0₄.7H₂O, 5×10⁻⁴FeSO₄ 7H₂O, 8 glycerol, 0.1 ampicillin, 3×10⁻³ (2×10⁻⁵ M) L-methionine, 0.2 (1.5×10⁻³ M) DL-norleucine (Gilles et al., J Biol. Chem. 263:8204-8209 (1988), produced a recombinant adenylate kinase where about 20% of the protein produced has all six of its methionines replaced with norleucine). In another method, as disclosed in U.S. Pat. No. 5,599,690, norvaline can be added to the culture medium to increase norleucine substitution (norleucine (0.25 g/L batch, 1.25 g/L feed) or norvaline (0.37 g/L batch, 1.25 g/L feed) supplementation produced up to 40% norleucine substitution in recombinant IL-2). It is theorized that norvaline is deamidated to form α-keto valerate, which can be converted into norleucine by the leucine biosynthetic pathway.

Alternatively, a two step fermentation, first in an amino acid-rich seed medium, then in a low amino acid fermentation medium (e.g., per liter: 10.90 g Na(NH₄)HPO₄.H₂O, 2.61 g K₂HPO₄, 1.92 g citric acid (anhydrous), 0.25 g MgSO₄.7H₂O; 0.66 g (NH₄)₂SO₄, 1.00 g yeast extract, 0.75 mL SAG4130, in R.O. water, later supplemented with a sterile micronutirent mix and cerelose) may be used to induce methionine substitution (Brunner et al., U.S. Pat. Nos. 5,698,418 and 5,622,845, showing a recombinant bovine somatotropin with up to 36% norleucine substitution at its four native methionines).

In some embodiments, the substituted, truncated, or substituted-truncated BMP-12-related proteins of the invention are produced by chemical synthesis, such as solid-phase peptide synthesis. Peptide synthesis is performed by means known in the art, including use of an automated peptide synthesizer. For a discussion of peptide synthesis, see, for example, John Howl Peptide Synthesis and Applications Humana Press; 1^{st} edition (2005), N. Leo Benoiton Chemistry of Peptide Synthesis CRC; first edition (2005), and U.S. Pat. No. 7,329,727.

The substituted, truncated, or substituted-truncated BMP-12-related proteins of the invention can also be produced using genetic engineering techniques known in the art. See, for example, Joseph Sambrook and David Russell, Molecular Cloning: A Laboratory Manual, Cold Spring Harbor Laboratory Press, 3rd edition (2001). For example, at least one of the “ATG” codons at nucleotides corresponding to nucleotides 253-255 and 364-366 of SEQ ID NO:2, which encode methionines 84 and 121 of SEQ ID NO:1, respectively, may be replaced with a non-methionine codon. In some embodiments methionine codons of a nucleic acid encoding a BMP-12-related protein are replaced with codons encoding leucine (CTT, CTC, CTA, CTG, TTA, TTG), isoleucine (ATT, ATC, ATA), valine (GTT, GTC, GTA, GTG), alanine (GCT, GCC, GCA, GCG), or phenylalanine (TTT, TTC). In more particular embodiments, the methionine codons are replaced with codons encoding leucine (CTT, CTC, CTA, CTG, TTA, TTG) or isoleucine (ATT, ATC, ATA). In some embodiments, only one of the codons encoding methionine residues corresponding to methionines 84 and 121 of SEQ ID NO:1 is replaced. In other embodiments, both of the codons encoding methionine corresponding to methionines 84 and 121 of SEQ ID NO:1 are replaced. When both codons are replaced, they may be replaced with the same codon or different codons.

Therefore, in some embodiments, the invention provides nucleic acids encoding the substituted BMP-12-related proteins of the invention. In certain embodiments, the codons encoding at least one of the amino acids corresponding to M84 or M121 of SEQ ID NO:1; or M75 or M112 of SEQ ID NO:3 or 4 are replaced. In more particular embodiments, codons encoding an amino acid corresponding to M72 of SEQ ID NO:3 or 4, and/or M31 of SEQ ID NO:4 are also replaced.

In some embodiments, the nucleic acid contains a degenerate sequence of nucleotides 4-390 of SEQ ID NO:2. In certain embodiments, the nucleic acid hybridizes under stringent hybridization conditions (for example, at least about 6×SSC and 1% SDS at 65° C., with a first wash for 10 minutes at about 42° C. with about 20% (v/v) formamide in 0.1×SSC, and with a subsequent wash with 0.2×SSC and 0.1% SDS at 65° C.) to SEQ ID NO:2 and encodes a substituted BMP-12-related protein with tendon and/or ligament-like tissue inducing activity. In some embodiments the invention provides a nucleic acid comprising a sequence that is at least 70%, 75%, 80%, 85%, 90%, 92%, 94%, 95%, 96%, 97%, 98%, 99%, or more, identical to nucleotides 4-390 of SEQ ID NO:2 and encodes a protein with tendon and/or ligament-like tissue forming activity.

In other embodiments, the nucleic acids of the invention encode a truncated BMP-12-related protein with tendon and/or ligament-like tissue forming activity. To make a truncated BMP-12-related protein, nucleotides encoding amino acids corresponding to, e.g., amino acids 1-27 and 129, 1-27, 1-22, 1-21, 1-18, 1-7, or 1-6 of SEQ ID NO:1; or 1-18 and 120, 1-18, 1-7, or 1-5 of SEQ ID NO:3 or 4 are deleted. The nucleic acids of the invention can be made by modification of wild-type BMP-12, BMP-13, or MP-52 by, for example, site-directed mutagenesis.

In some embodiments, the nucleic acids of the invention may be optimized to enhance protein expression levels in a particular host cell. Optimizations include, for example, codon optimization, modifications that affect mRNA stability, and modified translational initiation and termination sites. For additional discussion of ways to optimize recombinant protein expression, see, Gustafsson et al., Trends Biotechnol. 22:346-53 (2004) and Sorensen and Mortensen, J. Biotechnol. 115:113-28 (2005).

The nucleic acids of the invention may be contained in a vector. In some embodiments, the vector includes a selectable marker (for example, one or more genes encoding resistance to antibiotics such as ampicillin, tetracycline, ciprofloxacin, G418, or puromycin). In some embodiments, the vector includes a control sequence for driving the transcription and translation of the nucleic acids of the invention (for example, a galactose-inducible promoter or a constitutive promoter) and one or more origins of replication.

In some embodiments, the nucleic acids and vectors of the invention may be contained in an appropriate host cell. In some embodiments the host cell may be from, e.g., a mammal, e.g., human, mouse, rat, hamster, chimpanzee, or macaque; a fungus, e.g., fission or budding yeast; or bacterium, e.g., E. coli or B. subtilis, or P. fluorescens.

In some embodiments, truncated or substituted-truncated BMP-12-related protein can be produced by digestion of a BMP-12-related protein or substituted BMP-12-related protein. For example a full length, mature BMP-12-related protein or substituted BMP-12-related protein can be incubated with a protease, e.g., trypsin, for a period of time sufficient to produce a truncated or substituted-truncated BMP-12-related protein. For example a BMP-12-related protein (substituted or not) can be incubated with trypsin in, e.g., a buffered detergent solution, at an enzyme to substrate ratio of about 1:50, 1:100, 1:200, 1:500, 1:1000, 1:2000, or 1:4000 for a period of, e.g., about 1, 2, 4, 5, 10, 15, 20, 30 minutes, or more.

### Compositions and Carriers

The novel BMP-12-related proteins of the invention can be part of a composition. In some embodiments, the composition further comprises a BMP-12-related protein containing methionine residues in the positions corresponding to methionines 84 and 121 of SEQ ID NO:1 (“met-BMP-12-related protein”). In certain embodiments, the met-BMP-12-related protein comprises a sequence that is at least 70%, 75%, 80%, 85%, 90%, 92%, 94%, 95%, 96%, 97%, 98%, 99%, or more identical to SEQ ID NO:1, 3, or 4 and is able to induce formation of tendon and/or ligament-like tissue.

In some embodiments, the composition may comprise or consist essentially of BMP-12-related proteins, including substituted BMP-12-related proteins, truncated BMP-12-related proteins, substituted-truncated BMP-12-related proteins, met-BMP-12-related proteins, and combinations thereof. BMP-12-related proteins can make up at least about 0.1%, 1%, 5%, 10%, 15%, 20%, 25%, 50%, 70%, 80%, 90%, 95%, 99%, or more of the crude dry weight of the composition. In particular embodiments, substituted BMP-12-related proteins may make up at least about 0.1%, 1%, 5%, 10%, 15%, 20%, 30%, 40%, 50%, 75%, 80%, 85%, 90%, 95%, 99%, 99.9%, or more of the BMP-12-related proteins in the composition. In certain embodiments, the methionine residues of the BMP-12-related protein subunits in the composition can have a per residue substitution rate of at least about 1%, 5%, 10%, 15%, 20%, 25%, 30%, 35%, 40%, 50%, 60%, 70%, 80%, 90%, 95%, 99%, or more. In particular embodiments, the composition may include BMP-12, BMP-13, or MP-52, including combinations and heterodimers thereof, and further where the proteins may be substituted, truncated, or substituted-truncated.

In some embodiments, the composition is a fermentation product of a bacterium. In particular embodiments, the bacterium is grown in conditions selected from limited methionine, limited leucine, excess norleucine, and combinations thereof. In some embodiments, BMP-12-related proteins make up at least about 1%, 2%, 5%, 8%, 10%, 12%, 15%, 20%, 25%, 30%, 40%, 50%, or more of the total protein of the bacterium. In more particular embodiments, BMP-12-related proteins make up at least 10% of the total protein of the bacterium. In still more particular embodiments, BMP-12-related proteins make up about 10-24% of the total protein of the bacterium.

In some embodiments the composition may further comprise one or more pharmaceutical carriers. Suitable pharmaceutical carriers are selected based on the properties desired by a practitioner. For a general review of pharmaceutical carriers for BMPs, see, for example, Seeherman and Wozney, Cytokine Growth Factor Rev. 16(3):329-45 (2005). In general, carriers will need to retain the activity of the BMP-12-related proteins of the invention and be bioresorbable. Carrier molecules may advantageously increase the retention time of the BMP-12-related proteins at the treatment site. Additionally, carriers should allow for cell infiltration, without residual carrier interfering with healing.

Suitable carriers include buffers and solutions comprising solubilizing excipients and stabilizers, natural polymers, e.g., collagens, gelatin, hyaluronans, chitosans, silk, fibrin, alginate or agarose; artificial polymers, e.g., poly(α-hydroxy acid) polymers such as poly lactide or polyglycolide and their copolymers; and inorganic compounds, e.g., high- and low-temperature orthophosphates (such as calcium phosphates and sintered ceramics) and calcium sulfates.

In certain embodiments, the compositions of the invention contain additional growth factors, such as one or more additional bone morphogenetic proteins (BMPs). Descriptions of BMPs can be found in the following publications: BMP-2, BMP-3, BMP-4, BMP-5, BMP-6, and BMP-7 (disclosed, for example, in U.S. Pat. Nos. 5,108,922; 5,013,649; 5,116,738; 5,106,748; 5,187,076; and 5,141,905), BMP-8 (disclosed in PCTWO 91/18098), BMP-9 (disclosed in PCTWO 93/00432), BMP-10 (disclosed in PCTWO 94/26893) BMP-11 (disclosed in PCTWO 94/26892), BMP-12 and BMP-13 (disclosed in PCT WO 95/16035), BMP-15 (disclosed in U.S. Pat. No. 5,635,372), BMP-16 (disclosed in U.S. Pat. No. 6,331,612), MP-52 (disclosed in PCT WO 93/16099), and BMP-17 and BMP-18 (disclosed in U.S. Pat. No. 6,027,917). A reference to these proteins, should be understood to include variants, allelic variants, fragments of, and mutant BMPs, including but not limited to deletion mutants, insertion mutants, and substitution mutants. In particular, reference to any particular BMP should be understood to include N-terminal truncation fragments where at least 1, 3, 5, 7, 10, 15, 18, 20, 22, 25, 30, 35, or more residues have been removed from the N terminus of the mature protein. In particular embodiments, the composition includes heterodimers containing one subunit of a substituted, truncated, or substituted-truncated BMP-12-related protein of the invention and one subunit of another BMP. Heterodimers are descried in further detail in, e.g., WO 93/009229, incorporated by reference.

### Examples

### Example 1

### Discovery of Substituted BMP-12

During development fermentation conditions for the E. coli production of recombinant BMP-12, new species of BMP-12 were identified in late-eluting RP-HPLC peaks and confirmed in a peptide map.

Solubilized Inclusion Bodies (sIB) from a BMP-12 fermentation in E. coli were diluted to 0.2-0.5 mg/mL (estimated by A₂₈₀) in reduction buffer (5 M Guanidine HCl, 0.1 M Tris, pH 8.2), with a minimum dilution factor of 10. 1M DTT (dithiothreitol) was added to a final concentration of 10 mM to reduce BMP-12 to monomeric subunits. The reducing mixture was incubated at 40° C. for 30 minutes, and acidified with 10% TFA (Trifluoroacetic acid)(v/v) to a final concentration of 0.3% (v/v) TFA. Highly purified samples were diluted to 0.1 mg/mL in reduction buffer with a dilution factor of 10 and reduced by DTT as described above. The HPLC method for routine analysis and LC/MS analysis is as follows:

TABLE 2

| | Flow | | |
|---|---|---|---|
| Time | (mL/min) | % A | % B |
| 0 | 1 | 75 | 25 |
| 30 | 1 | 47 | 53 |
| 31 | 1 | 0 | 100 |
| 36 | 1 | 0 | 100 |
| 36.1 | 1 | 75 | 25 |
| 48 | 1 | 75 | 25 |

Columns: Poros R1/10, 4.6 × 100 mm, Applied Biosystems, product number 1-1014-2
Column temp: 40° C.
Sample temp: 4° C.
Detection wavelength: 214 nm
Injection: 100 μL (10 μg)
Run Time: 45 min
Mobile phase A: 0.1% TFA (w/v)
Mobile Phase B: 95% acetonitrile (v/v), 0.1% TFA (w/v)

The HPLC method for rapid in-process screening during fermentation is as follows:

TABLE 3

| | Flow | | |
|---|---|---|---|
| Time | (mL/min) | % A | % B |
| 0.01 | 2.5 | 75 | 25 |
| 12 | 2.5 | 47 | 53 |
| 12.4 | 2.5 | 0 | 100 |
| 14.4 | 2.5 | 0 | 100 |
| 14.5 | 2.5 | 75 | 25 |
| 19.2 | 2.5 | 75 | 25 |
| 24 | 2.5 | 75 | 25 |
| 25 | 0.1 | 75 | 25 |

Column: Poros R1/10 4.6 × 100 mm, Applied Biosystems product number 1-1014-26
Column temp: 40° C.
Flow rate = 2.5 mL/min
Load: 25 μg
Run Time: 18 minutes
Mobile phase A: 0.1% TFA (w/v)
Mobile Phase B: 95% acetonitrile (v/v), 0.1% TFA (w/v)

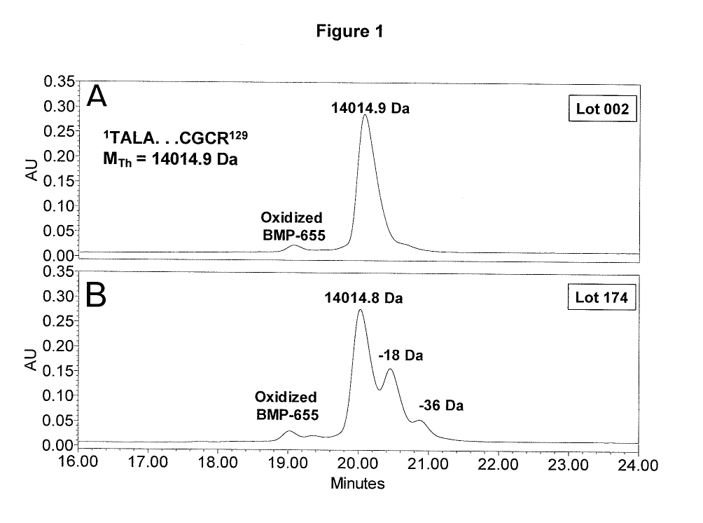

FIG. 1 shows reducing RP-HPLC profiles of highly purified BMP-12 with (Lot 174, FIG. 1B) or without (Lot 002, FIG. 1A) the two new peaks that elute just after the typical BMP-12 peak. Previous laboratory scale preparations of BMP-12 also lack the late-eluting peaks and show profiles similar to Lot 002.

### Example 2

### Fermentation, Not Purification, Produced New BMP-12 Species

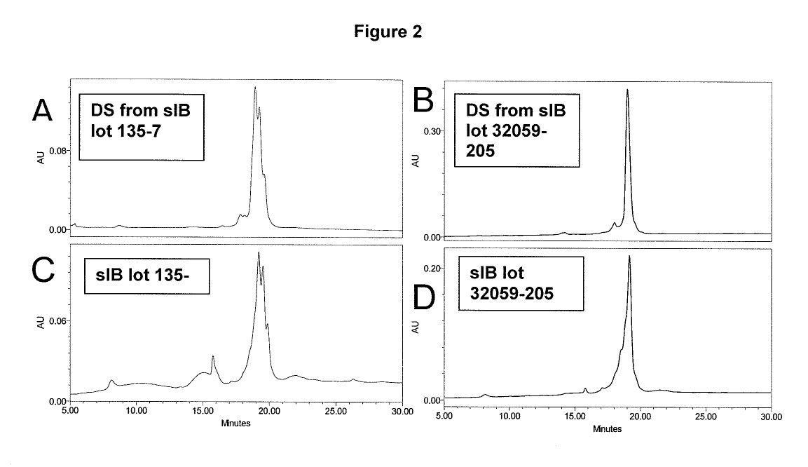

The new BMP-12 species described in the previous Example most likely resulted from the fermentation process and not subsequent purification. FIGS. 2A and 2C show that if the new species were already present in the sIB stage (FIG. 2A), they were not significantly removed by further purification (FIG. 2C). Conversely, FIGS. 2B and 2D show that when sIB preparations (FIG. 2B) did not contain the new species, they were also not present in a further purified sample (FIG. 2D). Similar results were obtained from several batches of sIB from different fermentations. Accordingly, the new BMP-12 species are likely to be a result of the fermentation process and not subsequent purification.

### Example 3a

### New BMP-12 Species Contain Substitutions at Methionine Residues

One of the purified BMP-12 materials containing the new BMP-12 species, lot 174, was selected to further characterize and identify the new species. Lot 148, an earlier prepared material, which did not show the new species in reducing RP-HPLC, was used as a control.

The reducing RP-HPLC profiles shown in FIG. 2 were further analyzed by coupling to a high-resolution Waters QTOF mass spectrometer (MS). Liquid chromatography/mass spectrometry (LC/MS) results show that the first major peak has an observed mass of 14014.8 Da, which is consistent with the theoretical mass of 14014.9 Da for a BMP-12 monomeric subunit. The two later eluting peaks containing the new BMP-12 species have mass differences of −18 Da and −36 Da relative to wild-type BMP-12, respectively. These later eluting peaks make up approximately 32% and 8% of total monomer species, respectively.

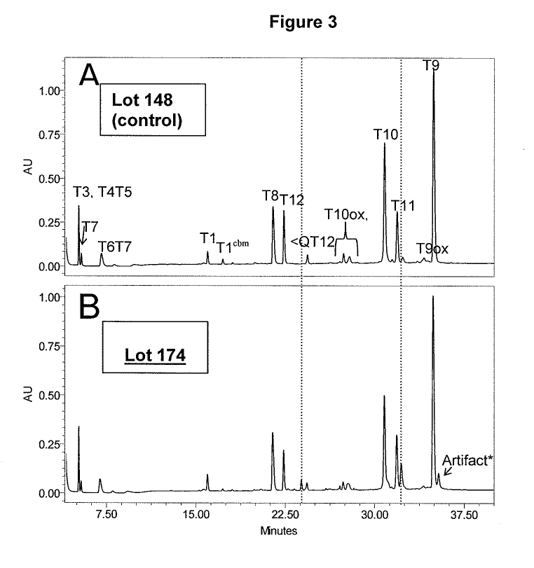

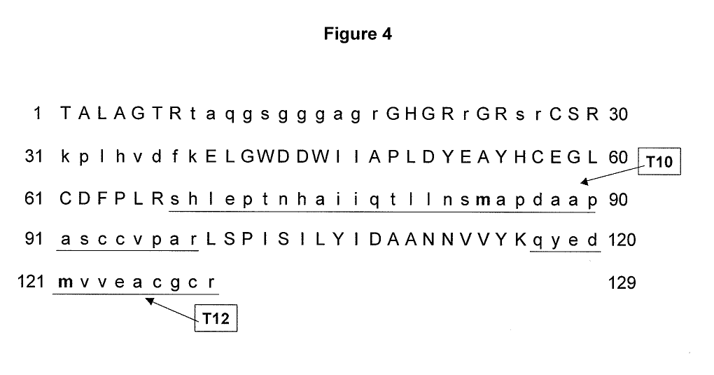

FIGS. 3A and 3B show the peptide maps of lots 148 (without the new species) and 174 (with the new species), respectively after alkylation and trypsination. A theoretical trypsin-peptide map of BMP-12 is shown in FIG. 4. Two new peaks (dashed lines) were present in the lot 174 map, while the T12 and T10 peptides showed a corresponding decrease in intensity. LC/MS peptide mapping also showed two new peaks, which localized the −18 Da mass differences to the two Met-containing peptides, T10 and T12. The high resolution QTOF mass spectrometer provided accurate mass differences of −17.949 and −17.957 Da, for the T10 and T12-derived peptides, respectively. The mass accuracy of the ESI-QTOF mass spectrometer allows for the characterization of the new BMP-12 species as containing substitution of methionine by leucine, isoleucine, or norleucine. The “artifact” peak in lot 174 was identified by mass spectrometry to be caused by incomplete reduction during sample preparation.

The exact mass difference for the substitution of leucine, isoleucine, or norleucine for methionine is −17.956 Da. Although mass values cannot differentiate between leucine, isoleucine, or norleucine, it is likely that the substituted amino acid is norleucine because overexpression of recombinant proteins in E. coli can lead to incorporation of norleucine in place of methionine (Tsai et al., Biochem. Biophys. Res. Comm. 156:733-739 (1988); Bogosian et al., J. Biol. Chem. 264:531-539, (1989)).

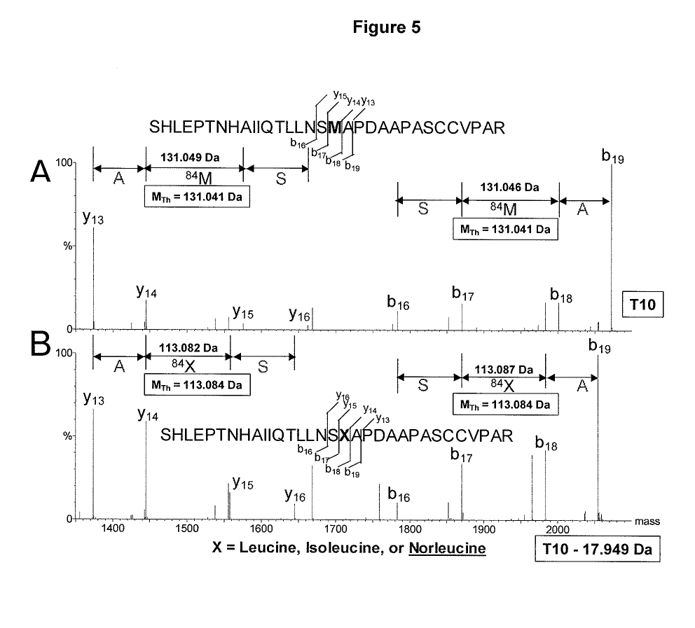

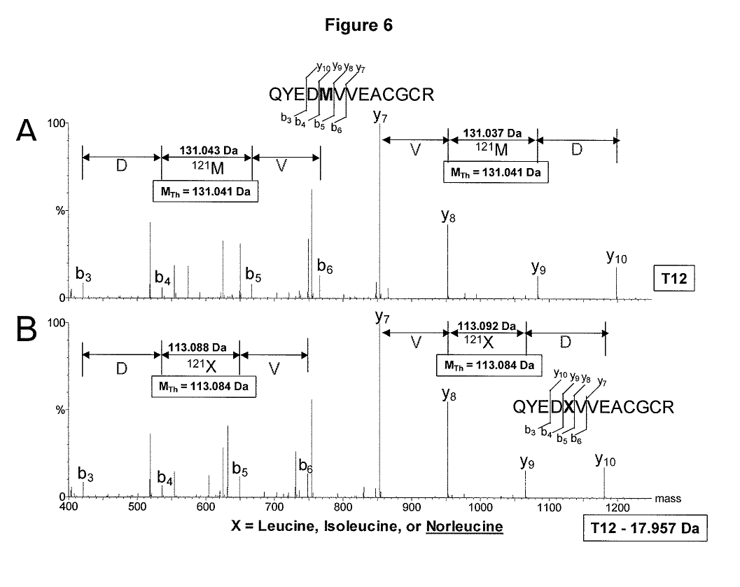

Methionine and norleucine containing T10 and T12 peptides were collected and then fragmented by nanoESI-QTOF MS/MS to confirm the substitution of norleucine for methionine (FIG. 5, FIG. 6). Approximately the same percentage of T10 and T12 peptides were in the norleucine-substituted form. This suggests that the there is no site-preference for the substitution of methionine with norleucine.

Based on the peptide mapping results, the two later-eluting peaks in the reducing RP-HPLC profile (FIG. 1) represent BMP-12 monomers with one (−18 Da) or both (−36 Da) methionines substituted with norleucine. In the disulfide-bonded dimeric form, up to four methionine to norleucine substitutions are possible. If the substitution at each methionine site on each monomeric subunit is random and there is no cooperativity or site preference, a 25% rate of substitution at each site (based on the relative peak areas of methionine and norleucine-containing peptides in the peptide map in FIG. 3) will lead to the following expected distribution of substituted monomeric subunits:
˜56% with no substitution (two methionines)
˜38% with a single substitution (one methionine and one norleucine)
˜6% with double substitution (two norleucines)

This predicted distribution roughly matches the actual distribution observed in the reducing RP-HPLC profile of FIG. 1, suggesting that substitution at the two sites was randomly distributed. Assuming that the various forms of substituted monomeric subunits (prior to refolding) behave identically to the non-substituted subunits during refolding, then the following distribution of dimeric species is expected:
˜32% with no substitutions (four methionines)
˜42% with one substitution (three methionines, one norleucine)
˜21% with two substitutions (two methionines, two norleucines)
˜5% with three substitutions (one methionine, three norleucines)
˜0.4% with four substitutions (four norleucines).

### Example 3b

### Substituted BMP-12 is Biophysically Similar to Wild-Type BMP-12

Although pure norleucine-substituted BMP-12 was not isolated, comparison between batches with and without significant amounts of substituted BMP-12 showed that substitution has no significant impact on 1) electrophoretic mobility (apparent size, by reducing and non-reducing SDS-PAGE and non-reducing SDS-CE), 2) aggregation, 3) disulfide knot formation (resistance to pepsin digestion), 4) folding (tryptophan fluorescence), or 5) in vitro biological activity (see Example 4).

### Example 4

### Substituted BMP-12 has Normal Bioactivity

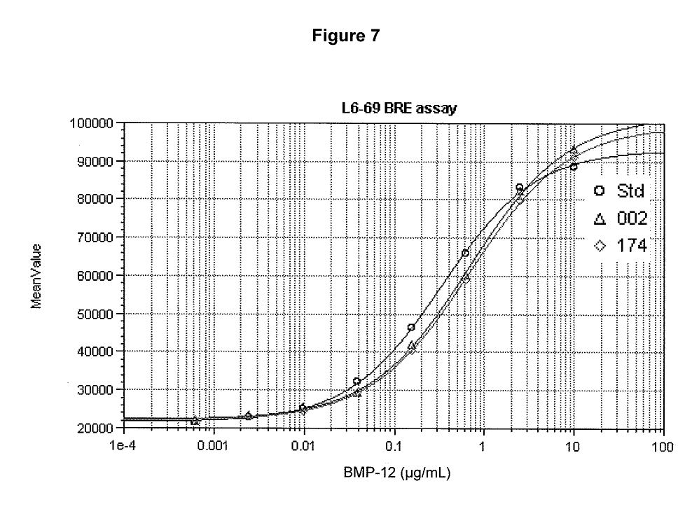

The in vitro biological activity of a BMP-12 batch containing significant substitution (lot 174) was compared to two batches of BMP-12 with low rates of substitution (lot 002 and lot 148 (Std); <5%, as detected by liquid chromatography/mass spectrometry) in the Bone Morphogenetic Protein Response Elements luciferase reporter gene bioassay (BRE-luc), described in detail in, for example, Kusanagi et al., Mol. Bio. Cell 11:555-65 (2000), incorporated by reference. The samples were diluted to 1 mg/mL in 50 mM acetic acid prior to the assay. These samples were sterile filtered prior to bioassay and the concentrations of the filtered samples were confirmed using A₂₈₀ values. All samples exhibited comparable activity in the bioassay (FIG. 7).

Lot 174 contained approximately 25% methionine to norleucine substitution at each site in the monomer. Based on reducing RP-HPLC, approximately 40% of the monomeric subunits contained at least one substituted methionine, which corresponds to about 70% of all BMP-12 dimers containing at least one substituted methionine. This level of substitution did not have any significant impact on the in vitro biological activity or other physical characteristics of BMP-12 tested.

### Example 5a

### Unsubstituted BMP-12 is Sensitive to Inactivation by Oxidation

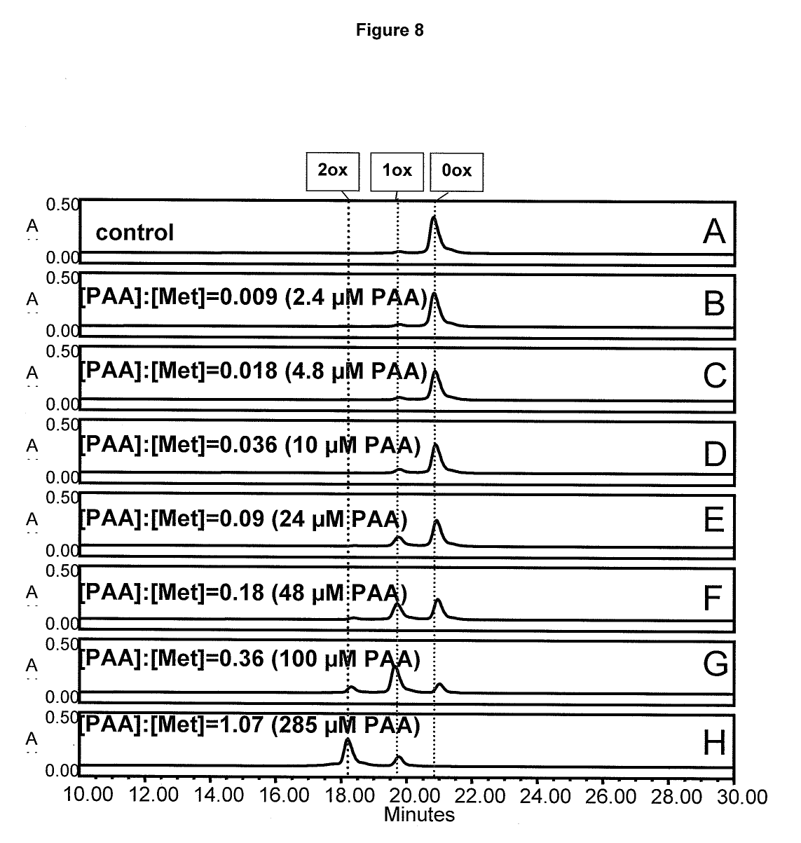

To investigate the effects of oxidation on BMP-12 in vitro biological activity, a batch of wild-type BMP-12 (<5% norleucine substitution) was incubated with varying concentrations of peracetic acid (diluted in formulation buffer) for two hours at room temperature, protected from light. Peracetic acid decomposes over time, but any residual peracetic acid that may have remained was removed by buffer exchange over Zeba Desalt spin columns (MWCO 7000). FIG. 8 shows the reducing RP-HPLC chromatograms of the oxidized samples. Reducing RP-HPLC separated the singly oxidized and doubly oxidized forms of the disulfide-reduced monomeric subunit (the peak identities were confirmed by liquid chromatography/mass spectrometry analysis). As peracetic acid levels increased, the proportion of oxidized forms of BMP-12 also increased. By comparing relative peak areas, it was apparent that doubly oxidized form showed an initial lag at low peracetic acid concentrations, but increased readily at higher peracetic acid concentrations.

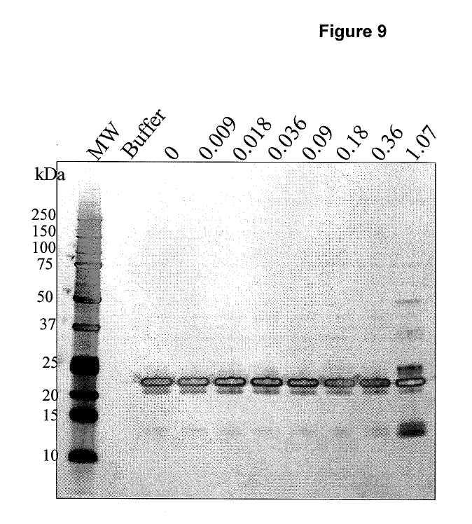

High levels of peracetic acid can lead to the oxidative cleavage of the interchain disulfide bond, causing the dissociation of BMP-12 dimer into inactive monomeric subunits. However, FIG. 9 shows that most of the dimer was intact in every sample. In the sample with the highest level of peracetic acid (far right lane), a slight increase of monomer and disulfide-scrambled dimer (a minor band migrating above the normal dimer band) were observed. Even in that sample, however, the majority of the protein was in the expected dimeric form and migrating at the same position as the control sample. Thus, minimal interference from oxidative cleavage of disulfide bonds was expected.

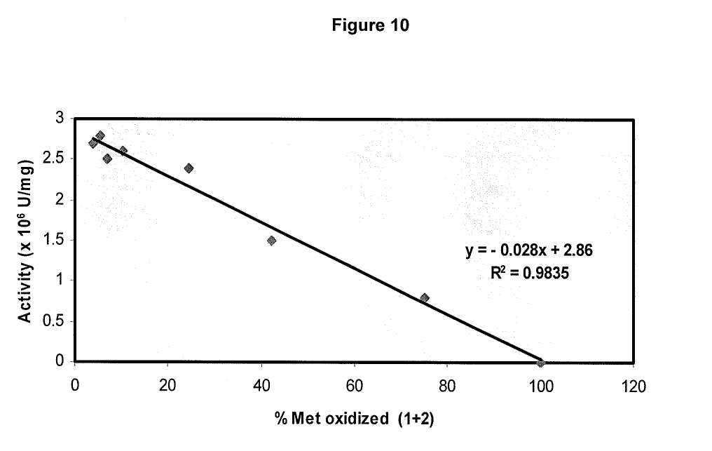

The biological activity of the peracetic acid-treated samples and the untreated control sample were measured by a BRE-luc bioassay. FIG. 10 shows the correlation between in vitro biological activity and levels of oxidation as measured by reducing RP-HPLC (FIG. 8). There was a direct negative correlation between the extent of methionine oxidation and in vitro biological activity.

As FIG. 10 shows, oxidation of either methionine residue in BMP-12 leads to reduced activity. Oxidation turns the hydrophobic side chain of methionine into a more hydrophilic one, which may result in a change in structure of the protein and/or affect interaction with, for example, receptors or another BMP-12 monomeric subunit. This suggests that hydrophobic side chains at the 84 and 121 positions are needed to maintain activity. Substitution of methionine with norleucine maintains the hydrophobic nature and the approximate size of the residue. Therefore it is reasonable to speculate that incorporation of norleucine in place of methionine would maintain both the structure and biological activity of BMP-12.

The sequence of rhBMP-2 also contains two methionine residues, one of which is conserved in BMP-12 (M121). The oxidation of methionine residues in rhBMP-2 by peracetic acid also leads to a decrease in activity. The fact that this residue is conserved between BMP-12 and BMP-2 underscores the importance of this particular methionine residue.

### Example 5b

### Substituted BMP-12 is Resistant to Oxidative Inactivation

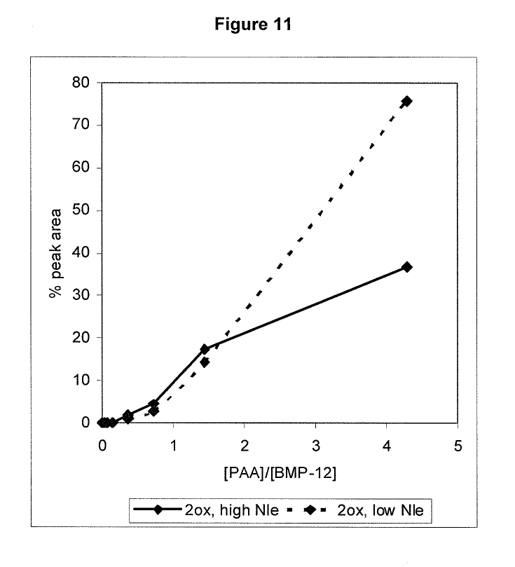

To investigate the effect of oxidation on substituted BMP-12, a pool of substituted BMP-12 batches containing 25-40% substitution at each methionine site was subjected to peracetic acid oxidation and compared to a batch with undetectable levels of substitution. At the highest level of peracetic acid, normal BMP-12 was ˜80% oxidized on both methionine residues, while only about 40% of the highly morleucine-subsituted pool was completely oxidized (FIG. 11). While the presence of norleucine did not appear to affect the rate of oxidation of any remaining methionine residues, it is expected that fully substituted BMP-12 would be fully resistant to oxidation.

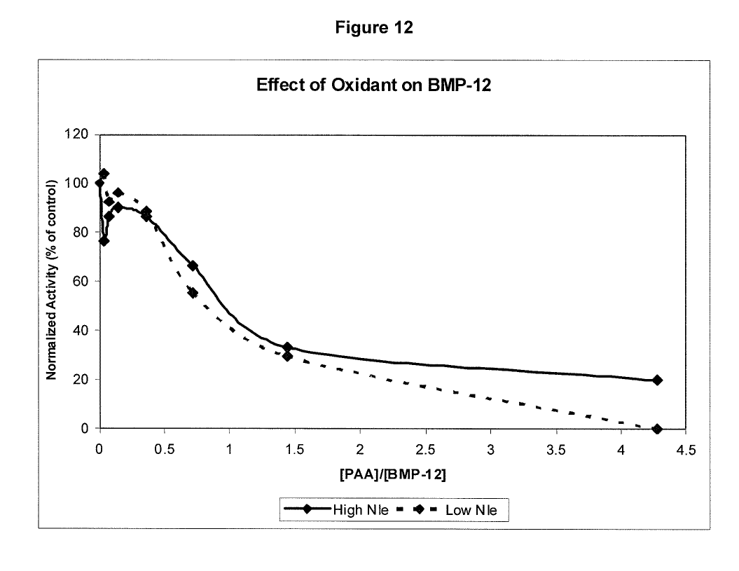

Next, the effect of oxidation on BMP-12 activity in vitro (as measured by a BRE-luc bioassay) in the highly norleucine-substituted batch pool was compared to a batch of BMP-12 without significant substitution. At relatively low levels of peracetic acid, the highly norleucine-substituted BMP-12 sample exhibited activity loss comparable to unsubstituted BMP-12. At higher levels of peracetic acid, however, the highly norleucine-substituted BMP-12 samples still maintained significant activity, while unsubstituted BMP-12 was completely inactive (FIG. 12). These results indicate that substituted BMP-12 is more resistant to oxidation-related inactivation.

### Example 6

### Fermentation Conditions

Modifications to basic fermentation conditions used and the level of substitution observed for different lots of BMP-12 are provided in Table 4. A prototrophic strain of E. coli was used for the production of BMP-12. Optical densities (OD) were measure on a Shimadzu UV 2401PC spectrophotometer.

The basic nutrition medium used in the E. coli fermentation (10 L or 60 L fermentation) process included (in g/L or ml/L): 6.8 g potassium phosphate monobasic, 2.0 g ammonium sulfate, 3.0 g trisodium citrate, 0.1 g CaCl₂2H₂O, 2.4 g MgSO₄ 7H₂O, 1.0 ml trace elements mixture (27.03 g ferric chloride, 1.29 g zinc chloride, 2.0 g sodium molybdate, 1.0 g calcium chloride, 1.27 g cupric chloride, 0.5 g boric acid, 2.86 g cobalt chloride, 100 ml HCl; final volume to one liter in distilled water), and optionally 2.0 to 4.0 g AmiSoy™ (soy protein hydrolysate). Amisoy™ (a source of amino acids) was not present in all fermentation conditions. After all ingredients were dissolved in water, the fermentor was sterilized by autoclaving for 60 minutes. After sterilization, the pH of the medium was adjusted under aseptic conditions to 7.0 and then 40% glucose stock solution (200 to 250 ml; final concentration 1.0 to 1.25 g/L) was added to 8 L of medium. In addition, either a commercially available Roswell Park Memorial Institute (RPMI) vitamins mix (1 ml/L) or yeast nitrogen base without amino acids (0.1 g/L) were sterile filtered and added to the autoclaved medium before it was inoculated with the recombinant E. coli strain carrying the plasmid for expression of the mature rhBMP-12 gene. The pH was controlled with concentrated ammonium hydroxide solution through a pH controller.

Post inoculation, the medium was aerated and maintained at 0.8 to 1.0 VVM to have dissolved oxygen saturation maintained at 20%, cascaded to the stirrer RPM. When the cell density (OD₆₀₀) reached about 15 to 18, 40% glucose stock solution feeding was initiated at 1.0 ml/minute (about 3.5 g/L/hour) until the cell density reached a desired value. When the OD₆₀₀ was between about 30 to 60, BMP-12 protein synthesis was induced by adding tryptophan to a final concentration of about 0.3 to 0.6 g/L and continuing the glucose feed for an additional 4 to 24 hours. The cells were harvested by centrifugation and mechanically broken open to isolate the inclusion body, which contains the BMP-12 protein.

TABLE 4

| | Per Site | Present in | | |
|---|---|---|---|---|
| | Misincorp. | Initial | | |
| Sample ID | Level | Medium | Met or Leu | Induction Conditions |
| LOT 174 | ~25% | 0.2% | None used in | 3 g tryptophan + 10 g yeast |
| (Purified | | Amisoy ™ | the medium or | extract prepared in about 200 ml |
| DS) | | yeast nitrogen | in the feed | |
| | | base (without | solutions | |
| | | aa) at 0.2 g/L | | |
| 135-7 | Significant (no | RPMI | None in the | 6 g tryptophan alone in about 150 mL |
| (Partially | peptide map | vitamins | medium or | 0.2 N NaOH solution |
| purified) | results | (1 ml/L) | feed solution | |
| | available) | | | |
| 181A-193 | ~25-40% | RPMI | None in the | 3.0 g tryptophan in 125 ml 0.2 N |
| (Partially | | vitamins | medium or in | NaOH; added twice, at 17 h and |
| purified) | | (1 ml/L) | the feed | 23 h |
| | | | solution | |
| Lots 148 | <5% | yeast nitrogen | None in the | 20X Induction solution contained |
| and 002 | | base (without | medium or in | per Liter: |
| (Purified | | aa) at 0.2 g/L | feed solution | Amisoy ™ 80 g; L-tryptophan 6.0 g; |
| DS) | | | | and yeast extract 20.0 g; yeast |
| | | | | nitrogen base without amino |
| | | | | acids, 4 g; 3 L of this added to |
| | | | | 60 L fermentation medium |

After inoculation of the batch that produced lots 148 and 002, a 10 g/L/hour glucose feed was started when OD₆₀₀ reached 8-10. When the OD₆₀₀ was close to 30 (at 8.7 hours), induction medium feed was applied and completed in 1.5 hours. The batch was harvested at 13 hours, when the final OD₆₀₀ was 55.0.

In other embodiments, the length of glucose feeds before induction of BMP-12 synthesis can vary. For example, in fermentation batches where Amisoy™ is present in the initial medium, glucose feed could be continued until the cell density OD₆₀₀ reaches about 30 or 60, in which case most of the amino acids present in the Amisoy™ would be metabolized. Accordingly, it is theorized, but not relied upon, that when gene expression is induced under these conditions, methionine substitution occurs because of, e.g., low levels of free methionine available to the cell and/or increased norleucine synthesis resulting from low-leucine-induced activation of the leucine pathway.

After the discovery of norleucine substitution in BMP-12, the E. coli 40% glucose feed was supplemented with methionine (0.1 M), leucine (0.1 M), or combination of methionine and leucine (0.05 M each). The resulting sIB preparations were partially purified and analyzed by reducing RP-HPLC and peptide mapping, coupled with ESI-QTOF MS. All three conditions resulted in undetectable or trace levels of BMP-12 substitution.

### Example 7a

### Identification of Truncated BMP-12 Species

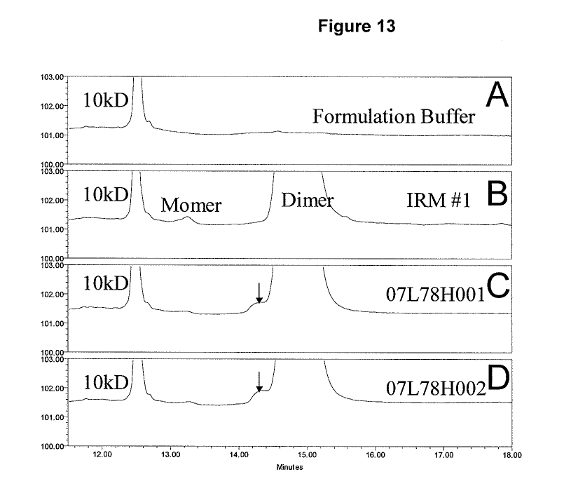

A new peak was observed in a non-reducing SDS-CE assay of some batches of purified BMP-12 (FIG. 13). The new peak was observed in two batches of BMP-12 (07L78H001 and 07L78H002, FIGS. 13C, 13D), but not in a previously purified reference batch (IRM #1; FIG. 13B). The new peak migrated just prior to the dimer peak, but later than the monomer peak. The apparent size of the new species was therefore less than 28 kDa but higher than 14 kDa. In addition, a new lower band was observed in reducing SDS-PAGE of these batches, but not in the reference batch. RP-HPLC fractionation and liquid chromatography/mass spectrometry analysis of these batches revealed that the new peak in non-reducing SDS-CE and the new band in reducing SDS-PAGE were both related to a 26 kDa, N-terminally truncated form of BMP-12. Liquid chromatography/mass spectrometry analysis also identified a 27 kDa, N-terminally truncated form of BMP-12 in certain preparations.

SDS-PAGE was further refined to better detect the truncated BMP-12 species. The truncated species were separated from full-length BMP-12 species by SDS-PAGE using 10% tricine gels. The non-reduced highly purified samples showed a faint low molecular weight (LMW) band (lower migration position), consistent with the non-reducing SDS-CE profiles. When the samples were reduced and alkylated, a LMW band was detected at higher total protein loads. The estimated molecular weight of the LMW band was about 2 kDa less than the main band.

Online RP-HPLC/MS analysis showed that one of the truncated species eluted in the latest ⅓ of the BMP-12 peak. To enrich the truncated species for further characterization, reduced or intact sample containing truncated species was fractionated by elution time during RP-HPLC. Reducing conditions were used in the analysis of monomeric subunits of BMP-12, while non-reducing conditions were used in the analysis of dimeric BMP-12. In order to allow higher loads necessary for fraction collection, a Poros R1/10 column was used.

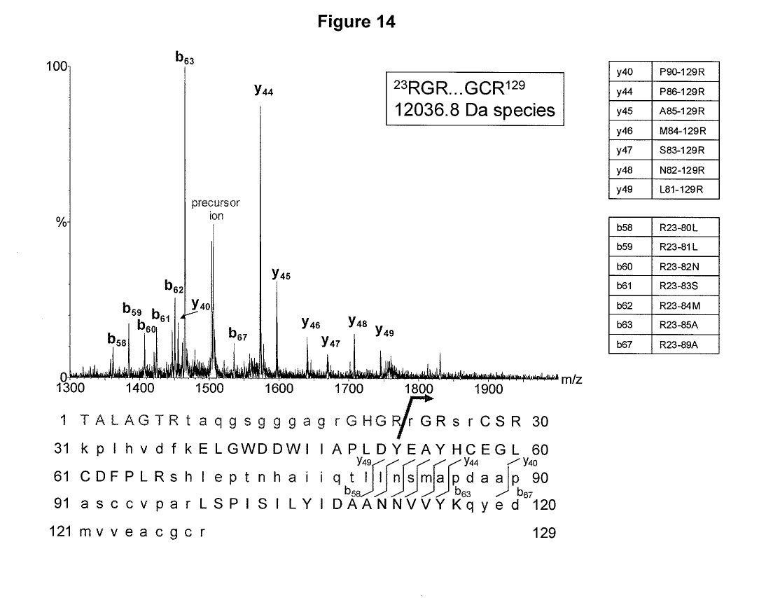

Two fractions of disulfide-reduced BMP-12 monomer were subjected to nanoelectrospray ionization QTOF-mass spectrometry (nanoESI QTOF-MS) and nanoESI QTOF MS/MS. MS mode was used to confirm that a late-eluting fraction contained truncated 12036.8 and 13344.1 Da species. The predominant charge state for the 12036.8 Da species was selected and fragmented by collision-induced-dissociation (CID) to sequence the species and confirm its identity as ²³RGR . . . GCR¹²⁹ (FIG. 14). The accurate masses determined for the b-type and y-type fragment ions comprising the sequence tag support the assignment of the NH₂-terminus as ²³RGR . . . GCR¹²⁹, which was based on the accurate mass analysis of unfragmented species. A similar analysis identified the 13344.1 Da species as ⁸TAQ . . . GCR¹²⁹.

RP-HPLC/MS was used to examine the presence of the truncated species in product pools collected throughout the purification process. The truncated species described above were detected throughout the purification process, without significant changes in abundance. Downstream purification did not remove these two truncated species to any significant degree, indicating they are structurally very similar to full-length BMP-12.

### Example 7b

### Enzymatically Truncated BMP-12 Shows Enhanced Activity

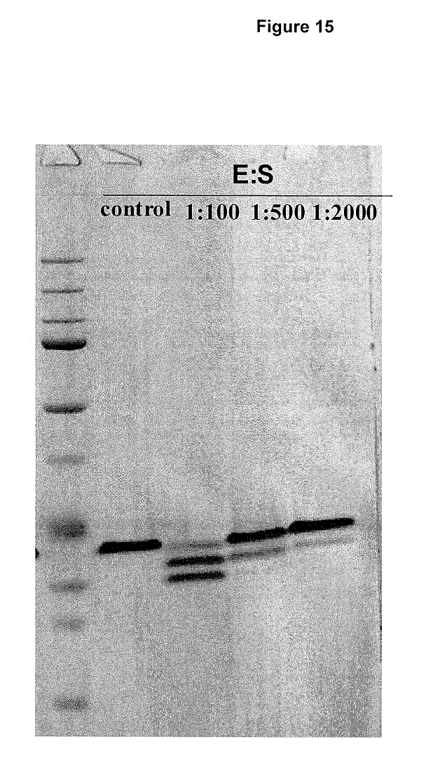

Truncated BMP-12 was intentionally produced by trypsin digestion of diluted, highly purified BMP-12 in a buffer-detergent solution that mimics the refold reaction (2% CHAPS, 0.1 M Tris, pH 8.4, 5 mM EDTA). Incubation of BMP-12 with very low levels of trypsin (E:S=2000) produced truncated species (FIG. 15), similar to those described in Example 7a (FIG. 14), within ten minutes at room temperature. At higher trypsin concentrations (such as E:S=100) or longer incubation times, further truncation can be observed.

Truncated species were produced by trypsin digestion of highly purified BMP-12 in a buffer-detergent solution that contains 0.2% Rapigest™ rather than CHAPS to allow liquid chromatography/mass spectrometry analysis. This analysis indicated that trypsin proteolysis produced BMP-12 having N termini of R7, R22, and R23, similar to those identified in Example 7a.

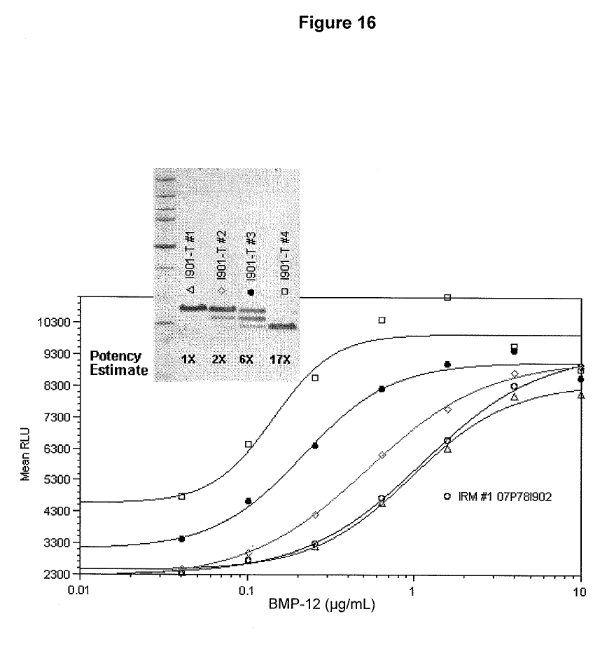

The trypsin-truncated BMP-12 species were tested in the BRE-luc bioassay and showed elevated in vitro bioactivity (FIG. 16). The in vivo activity of the truncated species was not tested.

An N-terminally truncated form of BMP-12 (²⁶SRC . . . GCR¹²⁹) was produced in E. coli, and found to induce tendon-like tissue in rat ectopic assays. The full-length BMP-12 molecule is also active in animal models. Since the truncations observed here were of intermediate size to full length BMP-12 and shorter truncated species of BMP-12—both of which are biologically active in vivo—the truncated species are expected to be biologically active in vivo too.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A substituted BMP-12-related protein comprising at least one amino acid substitution at a residue corresponding to methionine 84 and/or methionine 121 of SEQ ID NO:1; wherein the substituted BMP-12-related protein is capable of inducing the formation of tendon and/or ligament-like tissue.

2. The substituted BMP-12-related protein of claim 1, wherein the substituted BMP-12-related protein comprises a sequence that is at least 90% identical to SEQ ID NO:1.

3. The substituted BMP-12-related protein of claim 1, wherein the substituted BMP-12-related protein comprises a sequence that is at least 90% identical to SEQ ID NO:3.

4. The substituted BMP-12-related protein of claim 1, wherein the substituted BMP-12-related protein comprises a sequence that is at least 90% identical to SEQ ID NO:4.

5. The substituted BMP-12-related protein of claim 1, wherein the substituted BMP-12-related protein has an amino acid substitution at a residue corresponding to methionine 84 of SEQ ID NO:1.

6. The substituted BMP-12-related protein of claim 1, wherein the substituted BMP-12-related protein has an amino acid substitution at a residue corresponding to methionine 121 of SEQ ID NO:1.

7. The substituted BMP-12-related protein of claim 1, wherein the substituted BMP-12-related protein has an amino acid substitution at a residue corresponding to methionine 84 and an amino acid substitution at a residue corresponding to methionine 121 of SEQ ID NO:1.

8. The substituted BMP-12-related protein of claim 1, wherein the amino acid substitution at a residue corresponding to methionine 84 and/or methionine 121 of SEQ ID NO:1 is a residue selected from the group consisting of norleucine, leucine, isoleucine, valine, alanine, and phenylalanine.

9. The substituted BMP-12-related protein of claim 8, wherein the amino acid substitution at a residue corresponding to methionine 84 and/or methionine 121 of SEQ ID NO:1 is a residue selected from the group consisting of norleucine, leucine, and isoleucine.

10. The substituted BMP-12-related protein of claim 9, wherein the amino acid substitution at a residue corresponding to methionine 84 and/or methionine 121 of SEQ ID NO:1 is a norleucine residue.

11. The substituted BMP-12-related protein of claim 1, wherein the protein comprises at least one truncated subunit having an N-terminal truncation of between 1 and 27 amino acids.

12. The substituted BMP-12-related protein of claim 11, wherein the protein comprises at least one truncated subunit having an N-terminal truncation of between 1 and 22 amino acids.

13. The substituted BMP-12-related protein of claim 12, wherein the protein comprises at least one truncated subunit having an N-terminal truncation of between 1 and 18 amino acids.

14. The substituted BMP-12-related protein of claim 13, wherein the protein comprises at least one truncated subunit having an N-terminal truncation of between 1 and 7 amino acids.

15. A BMP-12-related protein comprising at least one truncated subunit having an N-terminal truncation of between 1 and 27 amino acids; wherein the BMP-12-related protein is capable of inducing the formation of tendon and/or ligament-like tissue.

16. The BMP-12-related protein of claim 15, wherein the truncated subunit has an N-terminal truncation of between 1 and 22 amino acids.

17. The BMP-12-related protein of claim 16, wherein the truncated subunit has an N-terminal truncation of between 1 and 18 amino acids.

18. The BMP-12-related protein of claim 17, wherein the truncated subunit has an N-terminal truncation of between 1 and 7 amino acids.

19. The BMP-12-related protein of claim 15, wherein the truncated subunit comprises a sequence that is at least 90% identical to SEQ ID NO:1.

20. The BMP-12-related protein of claim 15, wherein the truncated subunit comprises a sequence that is at least 90% identical to SEQ ID NO:3.

21. The BMP-12-related protein of claim 15, wherein the truncated subunit comprises a sequence that is at least 90% identical to SEQ ID NO:4.

22. The BMP-12-related protein of claim 15 having at least one amino acid substitution at a residue corresponding to methionine 84 and/or methionine 121 of SEQ ID NO:1.

23. A method of producing a substituted BMP-12-related protein comprising the steps of:
i) culturing a host cell comprising a nucleic acid sequence that encodes a BMP-12-related protein with tendon and/or ligament-like tissue inducing activity; and
ii) recovering a substituted BMP-12-related protein comprising at least one amino acid substitution at a residue corresponding to methionine 84 and/or methionine 121 of SEQ ID NO:1; wherein the substituted BMP-12-related protein is capable of inducing the formation of tendon and/or ligament-like tissue.

24. The method of claim 23, wherein the substituted BMP-12-related protein comprises a sequence that is at least 90% identical to SEQ ID NO:1.

25. The method of claim 23, wherein the nucleic acid comprises a sequence that is at least 90% identical to nucleotides 4-390 of SEQ ID NO:2.

26. The method of claim 23, wherein the BMP-12-related protein comprises a sequence that is at least 90% identical to SEQ ID NO:3.

27. The method of claim 23, wherein the BMP-12-related protein comprises a sequence that is at least 90% identical to SEQ ID NO:4.

28. The method of claim 23, wherein the host cell is a bacterium.

29. The method of claim 28, wherein the bacterium is E. coli.

30. The method of claim 28, wherein the bacterium is cultured in conditions selected from the group consisting of limited methionine, limited leucine, excess norleucine, and combinations thereof.

31. The method of claim 23, wherein the substituted BMP-12-related protein comprises at least one truncated subunit having an N-terminal truncation of between 1 and 27 amino acids.

32. A method of producing a BMP-12-related protein comprising a truncated subunit, comprising the steps of:
i) culturing a host cell comprising a nucleic acid that encodes a BMP-12-related protein with tendon and/or ligament-like tissue inducing activity; and
ii) recovering a BMP-12-related protein comprising at least one truncated subunit having an N-terminal truncation of between 1 and 27 amino acids; wherein the BMP-12-related protein is capable of inducing the formation of tendon and/or ligament-like tissue.

33. The method of claim 32, wherein the BMP-12-related protein comprises a sequence that is at least 90% identical to SEQ ID NO:1.

34. The method of claim 33, wherein the nucleic acid comprises a sequence that is at least 90% identical to nucleotides 4-390 of SEQ ID NO:2.

35. The method of claim 32, wherein the BMP-12-related protein comprises a sequence that is at least 90% identical to SEQ ID NO:3.

36. The method of claim 32, wherein the BMP-12-related protein comprises a sequence that is at least 90% identical to SEQ ID NO:4.

37. The method of claim 32, wherein the host cell is a bacterium.

38. The method of claim 37, wherein the bacterium is E. coli.

39. A nucleic acid encoding the substituted BMP-12-related protein of claim 1.

40. The nucleic acid of claim 39, wherein the amino acid substitution at a residue corresponding to methionine 84 and/or methionine 121 of SEQ ID NO:1 is a residue selected from the group consisting of leucine, isoleucine, valine, alanine, and phenylalanine.

41. A nucleic acid encoding the BMP-12-related protein of claim 15.

42. A composition comprising a substituted BMP-12-related protein comprising at least one amino acid substitution at a residue corresponding to methionine 84 and/or methionine 121 of SEQ ID NO:1; wherein the substituted BMP-12-related protein is capable of inducing the formation of tendon and/or ligament-like tissue.

43. The composition of claim 42, wherein the substituted BMP-12-related protein comprises a sequence that is at least 90% identical to SEQ ID NO:1.

44. The composition of claim 42, wherein the substituted BMP-12-related protein comprises a sequence that is at least 90% identical to SEQ ID NO:3.

45. The composition of claim 42, wherein the substituted BMP-12-related protein comprises a sequence that is at least 90% identical to SEQ ID NO:4.

46. The composition of claim 42, wherein the at least one amino acid substitution at a residue corresponding to methionine 84 and/or methionine 121 of SEQ ID NO:1 is selected from the group consisting of norleucine, leucine, isoleucine, valine, alanine, and phenylalanine.

47. The composition of claim 46, wherein the at least one amino acid substitution at a residue corresponding to methionine 84 and/or methionine 121 of SEQ ID NO:1 is a non-methionine residue selected from the group consisting of: norleucine, leucine, and isoleucine.

48. The composition of claim 42, wherein the composition is a fermentation product of a bacteria.

49. The composition of claim 48, wherein the bacteria expresses a protein encoded by a nucleic acid comprising a sequence that is at least 90% identical to nucleotides 4-390 of SEQ ID NO:2.

50. The composition of claim 48, wherein the bacteria is grown under conditions selected from the group consisting of limited methionine, limited leucine, excess norleucine, and combinations thereof.

51. The composition of claim 48, wherein the bacteria is E. coli.

52. The composition of claim 42, further comprising a BMP-12-related protein that comprises a sequence that is at least 90% identical to SEQ ID NO:1 and has the ability to induce the formation of tendon and/or ligament-like tissue.

53. The composition of claim 42, further comprising a BMP-12-related protein that comprises a sequence that is at least 90% identical to SEQ ID NO:3 and has the ability to induce the formation of tendon and/or ligament-like tissue.

54. The composition of claim 42, further comprising a BMP-12-related protein that comprises a sequence that is at least 90% identical to SEQ ID NO:4 and has the ability to induce the formation of tendon and/or ligament-like tissue.

55. The composition of claim 52, wherein the substituted BMP-12-related protein comprises at least about 1% of total BMP-12-related protein.

56. The composition of claim 55, wherein the substituted BMP-12-related protein comprises at least about 5% of total BMP-12-related protein.

57. The composition of claim 56, wherein the substituted BMP-12-related protein comprises at least about 10% of total BMP-12-related protein.

58. The composition of claim 57, wherein the substituted BMP-12-related protein comprises at least about 20% of total BMP-12-related protein.

59. The composition of claim 58, wherein the substituted BMP-12-related protein comprises at least about 50% of total BMP-12-related protein.

60. The composition of claim 52 consisting essentially of BMP-12-related proteins.

61. The composition of claim 52, comprising at least about 10% by weight of BMP-12-related proteins.

62. The composition of claim 61, wherein the composition is a fermentation product.

63. The composition of claim 42, further comprising a suitable pharmaceutical carrier.

64. A pharmaceutical composition comprising the substituted BMP-12-related protein of claim 1 and a suitable pharmaceutical carrier.

65. A pharmaceutical composition comprising the BMP-12-related protein of claim 15 and a suitable pharmaceutical carrier.

66. A method of treating a disease or defect of tendon or ligament-like tissue in a subject comprising administering an effective amount of the pharmaceutical composition of claim 64.

67. A method of treating a disease or defect of tendon or ligament-like tissue in a subject comprising administering an effective amount of the pharmaceutical composition of claim 65.

68. A product made by the method of claim 23.
